(19) United States
(12) Patent Application Publication
(10) Pub. No.: US 2021/0204381 A1
(43) Pub. Date: Jul. 1, 2021

(54) INTELLIGENT LIGHT FIXTURE MANAGEMENT METHOD BASED ON CHILDREN'S SLEEP

(71) Applicant: ANHUI GEEK DISCOVERY SCIENCE AND TECHNOLOGY CO., LTD., Hefei, Anhui (CN)

(72) Inventor: Longyun LI, Anhui (CN)

(73) Assignee: ANHUI GEEK DISCOVERY SCIENCE AND TECHNOLOGY CO., LTD., Hefei, Anhui (CN)

(21) Appl. No.: 16/307,478

(22) PCT Filed: Oct. 31, 2018
(86) PCT No.: PCT/CN2018/112985

§ 371 Date: Dec. 5, 2018

(30) Foreign Application Priority Data
Sep. 28, 2018 (CN) 201811140952.8

(51) Int. Cl. H05B 47/115

(52) U.S. Cl. CPC H05B 47/115

(57) ABSTRACT

The present application discloses a intelligent light fixture management method based on children's sleep, comprising: controlling a target light fixture to turn on, when the child is on the target bed; controlling the target light fixture to turn off, when the child is confirmed to fall asleep on the target bed; obtaining body parameter information and action information of the child when the child is sleeping; controlling the target light fixture to turn on, when the body parameter information or action information of the child when the child is sleeping indicates that the child is about to wake up. In this way, when the child is preparing to sleep in the bed, the light fixture is turned on to improve the security of the child before going to sleep, assisting that the child get to sleep quickly. After it's found that the child has fallen asleep, the light fixture is turned off to avoid the waste of electric energy. At the same time, the body parameter information and action information of the child when the child is sleeping are monitored, when the body parameter information or action information when the child is sleeping indicates that the child is about to wake up, the light fixture is turned on to ensure that the child will not feel lack of security because of afraid of darkness when waking up at night, thereby ensuring that children can continue to enter a normal state of sleep after waking up.

The present application claims priority to Chinese patent application NO.201811140952.8, filed to the Chinese Patent Office on Sep. 28, 2018, entitled “INTELLIGENT LIGHT FIXTURE MANAGEMENT METHOD BASED ON CHILDREN'S SLEEP”, the entire disclosure of which is incorporated herein by reference.

### BACKGROUND

### Technical field

The present application relates to the technical field of light fixture control, and in particular, to an intelligent light fixture management method based on children's sleep.

### Background art

With the increase in people's living standards and the pace of life, parents and children sleep differently in time and rhythm. Children often sleep in their own children's room, but for children, dark nights make children feel insecure. Parents usually turn on the lights to let the children sleep. However, the lights are turned on overnight, which seriously wasting electrical energy; if the parent turns off the light after the child falls asleep, when the child wakes up in the night, he/she will cry because of being afraid of blackness and lack of security, which will affect the child's normal sleep. How to ensure that children fall asleep quickly while ensuring that children have enough security when they wake up at night is an urgent problem in the field.

### SUMMARY OF THE INVENTION

Based on the technical problems existing in the background art, the present application provides an intelligent light fixture management method based on children's sleep.

The present invention provides an intelligent light fixture management method based on children's sleep, which comprises:

S1: controlling a target light fixture to turn on, when the child is on the target bed;

S2: controlling the target light fixture to turn off, when the child is confirmed to fall asleep on the target bed;

S3: obtaining the body parameter information and action information of the child when the child is sleeping;

S4: controlling the target light fixture turned on, when the body parameter information or action information of the child when the child is sleeping indicates that the child is about to wake up.

Preferably, in S1, the child is confirmed to be on a target bed by the pressure value detected through the pressure sensor preset on the target bed.

Preferably, in S2, confirming that the child falls asleep on the target bed, specifically comprises obtaining the child's current state via the intelligent wearable device worn by the child, to confirm that the child is asleep on the target bed.

Preferably, in S3, obtaining the body parameter information and action information when the child is sleeping, specifically comprises obtaining the body parameter information and action information of the child when the child is sleeping through an intelligent wearable device worn by a child.

Preferably, in S3, the body parameter information specifically comprises one or more of the heartbeat, the pulse, and the body temperature.

Preferably, in S4, the body parameter information when the child is sleeping indicates that the child is about to wake up, which specifically comprises that, the child is indicated to be about to wake up, when the child's heartbeat during sleeping exceeds a preset heartbeat threshold, and/or, when the child's pulse during sleeping exceeds the preset heartbeat threshold, and/or, when the child's body temperature during sleeping exceeds a preset body temperature threshold.

Preferably, in S3, the action information specifically comprises one or more of the arm motion amplitude, the wrist motion amplitude, and the trunk motion amplitude.

Preferably, in step S4, the action information when the child is sleeping indicates that the child is about to wake up, which specifically comprises that, the child is indicated to about to wake up , when the child's arm motion amplitude during sleeping exceeds a preset arm motion amplitude threshold, and/or the child's wrist motion amplitude during sleeping exceeds a preset wrist motion amplitude threshold, and/or the child's trunk motion amplitude during sleeping exceeds a preset trunk motion amplitude threshold.

In this application, when the child is on the target bed, the target light fixture is controlled to turn on, then when the child is confirmed to fall asleep on the target bed, the target light fixture is controlled to turn off, and the body parameter information and action information when the child is sleeping are obtained, when the body parameter information or action information of the child when the child is sleeping indicates that the child is about to wake up, the target light fixture is controlled to turn on. In this way, when the child is preparing to sleep in the bed, the light fixture is turned on to improve the security of the child before going to sleep, assisting the child to fall asleep quickly. After it's found that the child has fallen asleep, the light fixture is turned off to avoid the waste of electric energy. At the same time, the body parameter information and action information when the child is sleeping are monitored, when the body parameter information or action information when the child is sleeping indicates that the child is about to wake up, the light fixture is turned on to ensure that the child will not feel lack of security because of afraid of darkness when waking up at night, thereby ensuring that children can continue to enter a normal state of sleep after waking up.

### BRIEF DESCRIPTION OF DRAWING

FIG. 1 is a schematic flow chart of an intelligent light fixture management method based on children's sleep according to the present application

### DETAILED DESCRIPTION OF THE EMBODIMENTS

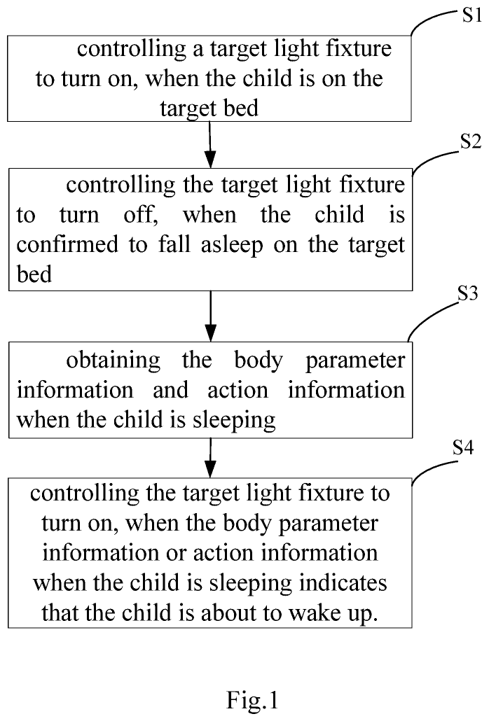

Referring to FIG. 1, the present application provides an intelligent light fixture management method based on children's sleep, which includes:

Step S1, controlling a target light fixture to turn on, when the child is on the target bed, specifically, the child is confirmed to be on a target bed by the pressure value detected through the pressure sensor preset on the target bed.

In a specific embodiment, the real-time pressure value of the child's bed is obtained through the pressure sensor set on the child's bed, the child is confirmed to be on the target bed, when the real-time pressure value is greater than the preset pressure threshold, at this point, the target light fixture is controlled to turn on. Further, if the child is confirmed to be on the target bed, the current time is obtained. and when the current time is within the night time interval preset by the user, the target light fixture is controlled to turn on.

Step S2, controlling the target light fixture turned off, when the child is confirmed to fall asleep on the target bed. Confirming that the child falls asleep on the target bed, specifically comprises obtaining the child's current state via the intelligent wearable device worn by the child, to confirm that the child is asleep on the target bed.

In a specific embodiment, taking the intelligent wearable device as an intelligent band or intelligent watch as an example, intelligent bands or intelligent watches can monitor the status of children wearing the intelligent bands or intelligent watch, which comprises sleep status, motion status and more.

Step S3, obtaining the body parameter information and action information when the child is sleeping, which specifically comprises obtaining the body parameter information and action information when the child is sleeping through the intelligent wearable device worn by the child. Wherein, the body parameter information specifically comprises one or more of the heartbeat, the pulse, and the body temperature; the action information comprises one or more of the arm motion amplitude, the wrist motion amplitude, and the trunk motion amplitude.

In a specific embodiment, taking the intelligent band or intelligent watch as the intelligent wearable device as an example, intelligent bands or intelligent watches may monitor the body parameter and the action parameter of the children wearing intelligent bands or intelligent watch to sleep, which comprises sleep status, motion status and more. Specifically, the body parameter information comprises one or more of the heartbeat, the pulse, and the body temperature, the action information comprises one or more of the arm motion amplitude, the wrist motion amplitude, and the trunk motion amplitude.

S4. controlling the target light fixture turned on, when the body parameter information or action information when the child is sleeping indicates that the child is about to wake up, which specifically comprises when the child's heartbeat during sleeping exceeds a preset heartbeat threshold, and/or, when the child's pulse during sleeping exceeds the preset heartbeat threshold, and/or, when the child's body temperature during sleeping exceeds the preset body temperature threshold, the child is indicated to be about to wake up.

Alternatively, when the arm motion amplitude of the child when the child is sleeping exceeds a preset arm motion amplitude threshold, and/or a wrist motion amplitude of the child when the child is sleeping exceeds the preset wrist motion amplitude threshold, and/or the trunk motion amplitude of the child when the child is sleeping exceeds a preset trunk motion amplitude threshold, the child is indicated to be about to wake up.

In a specific embodiment, when a child is having nightmares, it tends to be caused increasing of heartbeat, pulse, and body temperature of the child due to scare. The child is indicated to having nightmares and be about to wake up, when the child's heartbeat during sleep exceeds the preset heartbeat threshold, and/or, when the child's sleep pulse exceeds the preset heartbeat threshold, and/or, when the child's body temperature during sleep exceeds the preset body temperature threshold. At this point, the target light fixture is turned on, which ensure that children do not feel insecure due to darkness when they wake up at night.

Alternatively, when a child is sleeping and makes movement such as turning and the like, the child's arms, wrists, and trunk will swing to a large extent. The child is indicated to feel uncomfortable sleeping and is about to wake up, when the arm motion amplitude of the child when the child is sleeping exceeds the preset arm motion amplitude threshold, and/or the wrist motion amplitude of the child when the child is sleeping exceeds the preset wrist motion amplitude threshold, and/or the child's trunk motion amplitude during sleeping exceeds the preset trunk motion amplitude threshold.

In the present embodiment, when the child is on the target bed, the target light fixture is controlled to turn on, then when the child is confirmed to fall asleep on the target bed, the target light fixture is controlled to turn off, and the body parameter information and action information when the child is sleeping are obtained, finally, when the body parameter information or action information when the child is sleeping indicates that the child is about to wake up, the target light fixture is controlled to turn on. In this way, when the child is preparing to sleep in the bed, the light fixture is turned on to improve the security of the child before going to sleep, assisting the child to fall asleep quickly. After it's found that the child has fallen asleep, the light fixture is turned off to avoid the waste of electric energy. At the same time, the body parameter information and action information when the child is sleeping are monitored, when the body parameter information or action information when the child is sleeping indicates that the child is about to wake up, the light fixture is turned on to ensure that the child will not feel lack of security because of being afraid darkness when waking up at night, thereby ensuring that children can continue to enter a normal state of sleep after waking up.

The above is only the preferred embodiment of the present application, but the scope of protection of the present application is not limited thereto, and any equivalents or modifications of the technical solutions of the present application and the application concept thereof should be included in the scope of the present application within the scope of the technical scope of the present application.

What is claimed is:

1. A intelligent light fixture management method based on a child's sleep, characterized by comprising:
S1: controlling a target light fixture to turn on, when the child is on a target bed;
S2: controlling the target light fixture to turn off, when the child is confirmed to fall asleep on the target bed;
S3: obtaining a body parameter information and action information of the child when the child is sleeping;
S4: controlling the target light fixture to turn on, when the body parameter information or action information of the child when the child is sleeping indicates that the child is about to wake up.

2. The intelligent light fixture management method based on a child's sleep according to claim 1, characterized in that, in step S1, the child is confirmed to be on the target bed by a pressure value detected through a pressure sensor preset on the target bed.

3. The intelligent light fixture management method based on a child's sleep according to claim 1, characterized in that, in step S2, confirming that the child falls asleep on the target bed specifically comprises obtaining a current state of the child through an intelligent wearable device worn by the child, to confirm that the child is asleep on the target bed.

4. The intelligent light fixture management method based on a child's sleep according to claim 1, characterized in that, in step S3, obtaining the body parameter information and action information of the child when the child is sleeping specifically comprises obtaining the body parameter information and action information of the child when the child is sleeping through an intelligent wearable device worn by a child.

5. The intelligent light fixture management method based on a child's sleep according to claim 1, characterized in that, in step S3, the body parameter information specifically comprises one or more of heartbeat, pulse, and body temperature.

6. The intelligent light fixture management method based on a child's sleep according to claim 5, characterized in that, in step S4, the body parameter information of the child when the child is sleeping indicates that the child is about to wake up specifically comprises: the child is indicated to be about to wake up, when a heartbeat of the child during sleeping exceeds a preset heartbeat threshold, and/or, when a pulse of the child during sleeping exceeds a preset heartbeat threshold, and/or, when a body temperature of the child during sleeping exceeds a preset body temperature threshold.

7. The intelligent light fixture management method based on a child's sleep according to claim 1, characterized in that, in step S3, the action information specifically comprises one or more of an arm motion amplitude, a wrist motion amplitude, and a trunk motion amplitude.

8. The intelligent light fixture management method based on a child's sleep according to claim 7, characterized in that, in step S4, the action information of the child when the child is sleeping indicates that the child is about to wake up specifically comprises: the child is indicated to about to wake up, when the arm motion amplitude of the child when the child is sleeping exceeds a preset arm motion amplitude threshold, and/or the wrist motion amplitude of the child when the child is sleeping exceeds a preset wrist motion amplitude threshold, and/or the trunk motion amplitude of the child exceeds a preset trunk motion amplitude threshold.
